(19) United States
(12) Patent Application Publication
(10) Pub. No.: US 2008/0208119 A1
(43) Pub. Date: Aug. 28, 2008

(54) Safety Syringe With Retractable Needle Utilising Spring Clip On Plunger

(75) Inventors: Graeme Francis Walton, New South Wales (AU); Allan Walsh, New South Wales (AU)

Correspondence Address: BROOKS KUSHMAN P.C., 1000 TOWN CENTER, TWENTY-SECOND FLOOR, SOUTHFIELD, MI, 48075, US

(73) Assignee: GLOBAL MEDISAFE HOLDINGS LIMITED, The Hill, NSW (AU)

(21) Appl. No.: 11/910,553

(22) PCT Filed: Mar. 10, 2006
(86) PCT No.: PCT/AU2006/000319

§ 371 Date: Oct. 3, 2007

(30) Foreign Application Priority Data
Apr. 4, 2005 (AU) PCT/AU2005/000491
Jan. 23, 2006 (AU) 2006900313

(51) Int. Cl. A61M 5/32

(52) U.S. Cl. 604/110; 604/196

(57) ABSTRACT

A safety syringe having a retractable exchange needle is disclosed. The safety syringe (11) has a retractable needle (14) incorporated therein. The syringe barrel (12) has a plunger (13) slideably located therein, with piston means 26 located towards the end of the plunger remote from the end of the barrel from which the plunger extends. The needle (14) has a needle hub (15) integral with the needle. The plunger has engagement means in the form of a clip (22) having barbed means (24) associated therewith and located on its end to engage with the needle hub in order to retract the needle from an in use position into the syringe barrel after the syringe has been used.

### TECHNICAL FIELD

This invention relates to a safety syringe having a retractable needle for use in the medical or dental profession or in personal drug administration so that the physician, surgeon or other needle operator might be protected from injury by the needle after its use. In particular, it relates to means for causing retraction of a needle in safety syringes by engaging the plunger of the syringe with the hub of the needle.

### BACKGROUND

The danger of injury and possible infection from the HIV or hepatitis B virus to medical practitioners using needles in the normal course of their business is well documented.

Furthermore, persons who are in the habit of administering drugs to themselves run a severe risk of contacting either of the specified viruses, or indeed contacting other viruses if a needle, once used, is reused in an unsterilised form. There are also added dangers where the needle in the syringe is required to be replaced.

There have been many proposals aimed at reducing the number of so-called needle-stick injuries and various attempts have been made to provide a safe system for disposal of such needles once used, but such prior proposals have had deficiencies.

### OBJECT OF THE INVENTION

It is an object of the present invention to provide a syringe having a retractable surgical needle.

At the very least the invention provides an alternate means for retracting a needle by accommodating the needle of a used syringe in the barrel thereof to protect against accidental injury arising from unwanted contact with the exposed needle once the syringe has been used.

Although the following description generally refers to a syringe of conventional size, no such limitation is intended thereby, and reference to a syringe is meant to encompass any other needle/syringe combination including slimline syringes, where, by suitable adaptation, the invention may also be usefully applied.

It should also be understood that the invention is equally applicable to syringes where the needle is initially held directly in the barrel of the syringe as well as so-called exchange needle syringes where the needle is held in a removable gland, so that the needle gland assembly is replaceable, if already in place, or otherwise may be assembled by the user according to such requirements as gauge of needle etc.

### DISCLOSURE OF THE INVENTION

The invention in one broad form provides a safety syringe incorporating a retractable needle wherein the syringe has a syringe barrel which has a plunger slideably located therein, with piston means located towards the end of the plunger remote from the end of the barrel from which the plunger extends, a needle having a needle hub integral with the needle, wherein the plunger has engagement means in the form of a clip having barbed means and located on its end to engage with the needle hub in order to retract the needle from an in use position into the syringe barrel after the syringe has been used.

The needle hub may be held in a removable gland prior to be being withdrawn or it may be held directly by the barrel of the syringe.

Preferably the needle is housed in a gland to provide an exchange needle facility.

In the case where a gland is utilised, preferably the gland is fitted to the end of the barrel by threaded means.

Preferably the clip is a cylindrical clip fitted about a shank or a stem on the end of the plunger.

The clip may be provided with one or more barbs.

Each barb is preferably formed from a portion of the wall of the clip which is cut so as to form a flap which when bent provides a barbed element. Preferably the barb is formed so as to have a sharp point.

Preferably, a dimple is formed along a portion of the fold line by which the flap is bent to form the barb. This provides greater strength to the fold line preventing the barb from being flattened into alignment with the wall of the clip thereby assisting in preventing the barb from being bent back to become inoperative. With advantage, a corresponding groove may be provided in the inner region of the needle hub to accommodate the dimple.

Preferably the barrel is provided with a locking bush, which when the plunger is fully retracted with the needle attached, the plunger is prevented from being removed from the barrel or depressed again, thereby rendering the syringe safe and unusable.

Preferably a break point is provided on the plunger so that the plunger may be snapped off retaining the needle safely within the syringe barrel after use.

Preferably the needle is protected by a needle cap prior to use.

Prior to use, the needle cap protects the needle from damage and also acts as a safety cap for the medical staff using the syringe preventing needle stick injuries. The presence of the needle cap also means that the needle assembly may be conveniently changed without risk of needle stick injury which might occur if the needle were unprotected.

Where a needle gland is employed, the gland is preferably held in the needle cap by interference fit. Preferably there are matching splines on the outside of the needle gland that match with splines on the inside of needle cap.

The needle hub is pushed into the needle gland and is held in position by an interference fit. Sealing is achieved by the use of an O-ring located between the needle hub and the gland.

Thus in a preferred form of the invention, the plunger has built into its end a clip to engage with the needle hub after the plunger has expelled the fluid from the syringe, ie after completing an injection. When the two mating parts are pushed together, the plunger is then pulled back causing the used needle hub inclusive of the steel needle to come out of the needle gland or the body of the syringe as the case may be. The needle is now connected to the end of the plunger and can be withdrawn into the body of the syringe. Preferably the plunger has a snap point created by way of reduced diameter near the piston. When the plunger is snapped off, the syringe is rendered inoperative.

A split threaded bush is preferably located around the body of the plunger and pushed into the barrel end and is held in position by way of an interference fit and a matching groove. Preferably, the split bush acts as a locking means to secure the plunger against further use.

In a preferred embodiment, a stainless steel pressed clip is made from sheet metal and pushes over a stem or shaft on the end of the plunger. The stainless steel clip is made in the form of a “C” section that clamps around the stem approximately 5 mm long with a barb being formed on one side to interact with the inner wall of the needle hub at the completion of injection. After the needle is removed from the patient, the plunger is pulled back, the stainless steel barb on the plunger having locked into the needle hub causing the needle hub and thus the needle to retract into the body of the barrel rendering the needle safely stored from human harm. The separation of the needle from the plunger cannot be achieved and when the plunger is retracted and locked into the threaded split bush and the plunger is snapped off, the syringe is locked and is unable to be used again. It will be appreciated that the protrusion on the plunger has an interference fit to the inside of the needle hub and this makes it obvious when filling the syringe prior to injection at which point the plunger “bottoms” in the syringe barrel. However, after injection a more powerful stroke is required to overcome the initial resistance in order to actually lock the needle hub onto the plunger.

With advantage a depth stop may be provided on the plunger to prevent the plunger from coming into mating contact with the needle hub until it is required to do so, ie at the completion of the injection.

Preferably the cavity in the needle hub which receives the stem of the plunger with the barbed clip attached, is shaped so as to improve the efficiency of the barb in engaging with the hub. This is best explained in connection with the drawings.

### BRIEF DESCRIPTION OF THE DRAWINGS

The invention may be better understood from the following non-limiting description of preferred embodiments, in which:

FIG. 1 is a cross sectional view of one embodiment of the invention utilising a locking bush according to one aspect of the invention in which a snap lock split bush is utilised to retain the plunger after use;

FIG. 2 is a cross sectional view of the plunger utilised in the embodiment of FIG. 1,

FIG. 3 is a cross sectional view of the barrel utilised in the embodiment of FIG. 1,

FIG. 4 is a detailed cross sectional view of the end of the plunger of FIG. 2, showing a barbed clip attached,

FIG. 5 is a cross sectional view of the needle hub showing the shaping which has been found more beneficial,

FIG. 6 is a detailed cross sectional view of the end of the barrel of the syringe of FIG. 1, which accommodates a needle housed initially in a gland,

FIG. 7 is a cross sectional view of the gland which accommodates the needle hub,

FIG. 8 shows in plan view a spring clip according to one embodiment of the invention for use with the syringe illustrated in FIGS. 1 to 8,

FIG. 9 is a front elevation of the spring clip of FIG. 9

FIG. 10 is a side elevation of a spring clip of FIG. 9, showing the barb protruding,

FIG. 11 is a cross sectional view of a clip on a plunger stem mating with a needle hub having a simple, ie cylindrical profile,

FIG. 12 is a cross sectional view of a preferred embodiment of the invention, where the inner profile of the needle hub co-operates with the barb of the clip to advantage.

### DETAILED DESCRIPTION OF THE DRAWINGS

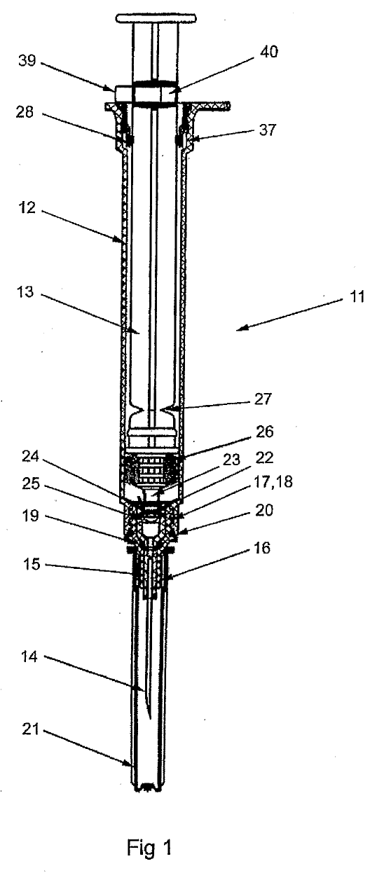

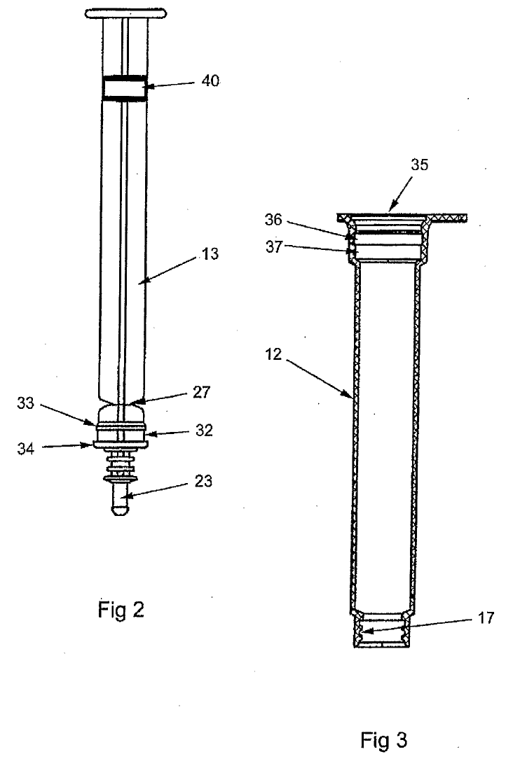

Referring generally to FIGS. 1 to 7 and especially FIG. 1, there is shown a syringe according to one aspect of the invention generally referenced 11, which comprises a syringe body or barrel 12, a plunger 13 slideable therein and a needle 14. The needle 14 is provided with a hub 15. The plunger 13 and barrel 12 are shown separately in FIGS. 2 and 3 for greater clarity.

The needle hub 15 is located by interference fit in a gland 16 which in this embodiment is screwed into the end of the barrel 12 utilising an internal female thread 17 on the inside of the end of the barrel 12, and a male thread 18 on the gland 16. In this embodiment the needle assembly comprising the needle and hub 14/15 and gland 16 may be replaced or exchanged (or indeed fitted) prior to use of the syringe so to provide flexibility of choice with respect to needle gauge for example.

It will also be appreciated that in the alternative, the gland 16 could be screwed onto the outside of the barrel 12, for example in slimline syringes. These embodiments allow for replacement of the needle assembly in a so-called exchange needle. On the other hand, the needle and hub 14/15 may be utilised directly in the end of the barrel 12 in integral arrangement, without a gland 16, in other embodiments of the invention, where there is no need to exchange the needle 4.

A small seal 19 is provided to seal between the needle hub 15 and gland 16, whilst a second larger seal 20 is provided to seal between the gland 16 and the barrel 12

The needle 14 is protected by a cover 21 prior to use.

Plunger 13 is provided with clip means 22 located on a stem 23 at the end of the plunger 13. The clip 22 is in the form of a spring clip and has a barb 24 which engages with the inner region 25 of the needle hub 15 when the plunger 13 is brought into contact with the needle hub 15 at the end of the injection.

Plunger 13 is also provided with a piston region 26 about its lower periphery which seals against the inside of the syringe body 12, when inserted therein.

A break groove or weaker region 27 is provided at a suitable location on the plunger 13, so that it may be broken off after it has retracted the needle 14 into the barrel of the syringe 12, that is to say, after use as described below.

A locking bush 28 is located in the upper region of the barrel 12.

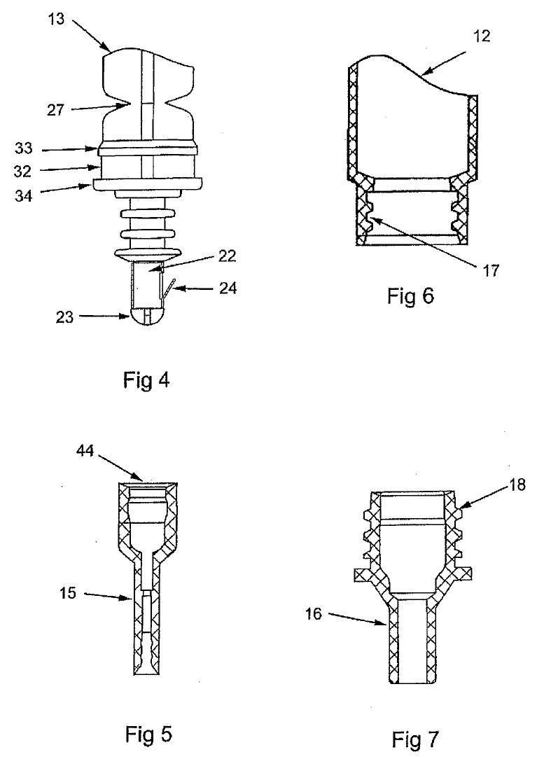

Details of the spring clip 22 and the components with which it co-operates to allow the plunger 13 to retract the needle 14 are shown in detail in FIGS. 4 to 10.

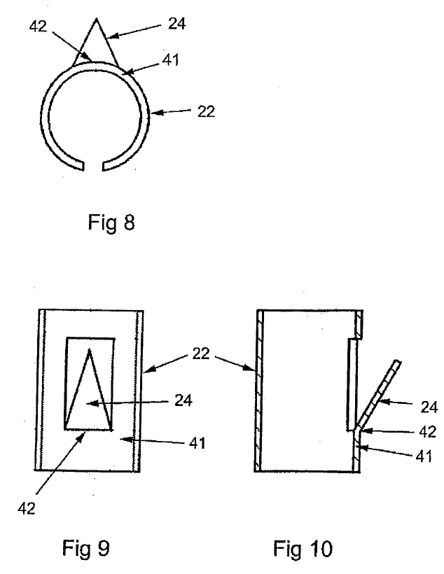

FIG. 4 shows the end of the plunger 13 with the clip 22 located thereon. The stem 23 on the end of the plunger 13 is fitted with the stainless steel clip 22 (shown in detail in FIGS. 8 to 10). The clip 22 is in the form of a semi-circular spring clip which fits about the stem 23, the shape of which is apparent in FIG. 8. The clip has barb 24 formed in the wall 41 of the clip by cutting, pressing or punching so as to form a flap which is bent along line 42 to form the barb 24. A dimple (not shown) may be formed in the fold line 42 to provide additional strength and resilience to the barb 24 being pushed back into alignment with the wall 42 of the clip 22. As shown in FIGS. 8 to 10, the barb 24 is preferably pointed, although other profiles may be contemplated

FIG. 5 shows in detail the inner profile of bore 44 of the needle hub 15, the nature of the profile of bore 44 being specifically discussed in conjunction with FIGS. 12 and 13 below.

FIG. 6 shows in detail the end of the barrel 12 and gland 16. Gland 16 screws into the end of the barrel 17 utilising threads 17, 18. Gland 16 receives needle and needle hub 14, 15 and holds the hub 15 by virtue of interference fit therewith.

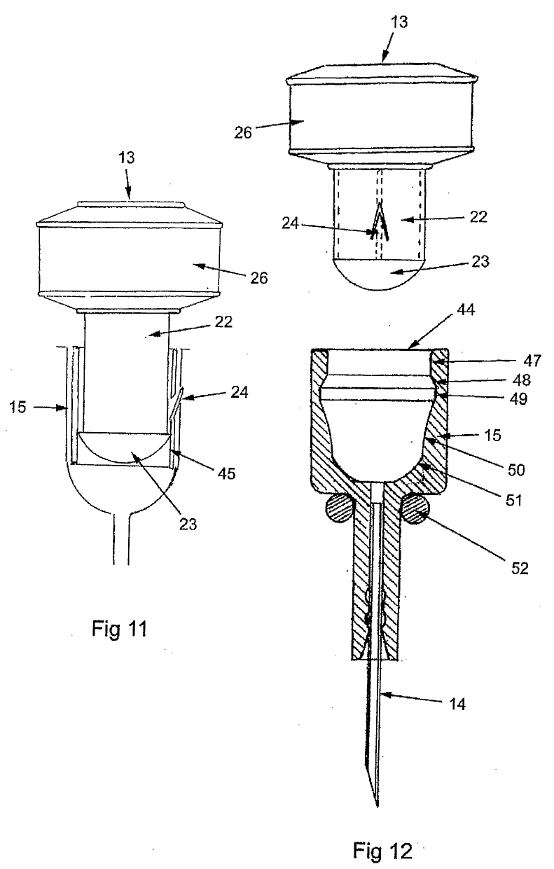

Turning to FIGS. 11 and 12, FIG. 11 seeks to show how a cavity in the hub 15 having simple cylindrical shape 45 is less satisfactory than one designed as in FIG. 13 (being a detail of bore 44 as seen in FIG. 5). Because the bore 45 of FIG. 12 needs to be slightly larger in diameter than the assembled stem/clip 22, 23 in order to allow the barb 24 to enter freely, when the plunger 13 is retracted, one of two problems is likely to occur. Either the barb 24 on the clip 22 fails to bite into the inner wall of bore 45 of the needle hub 15 so that retraction is less likely to occur, or if the barb 24 does bite into the wall 45 of the needle hub 15, the spring clip 22 expands when the plunger 13 is retracted thereby allowing the plunger 13 to slide through the centre of the expanded spring clip 22 as depicted in FIG. 11.

On the other hand, turning to FIG. 12, the barb 24 on the split cylinder of the spring clip 22 is pressed within the wall 44 of the cylinder and expands outwards from the surface at 33°. The spring clip 22 is pressed from sheet steel and the clip 22 is located on the plunger 13 on a stem 23 located below the piston 26. The needle hub 15 is provided with a shaped bore 44 which has a first region 47 of sufficient diameter to provide clearance for the plunger 13 and spring clip 22, along with the protruding barb 24. Without this clearance the barb 24 would be de-activated. The second region 48 is tapered in order to cause the barb 24 on the spring clip 22 to bite into the wall 44 of the needle hub 15. Without this taper the barb 24 would often fail to activate.

The third region 49 allows the barb 24 on the spring clip 22 to settle in to the correct position prior to retraction of the plunger/needle hub 13, 15 and it allows for any variation in length to be and accommodated.

The fourth region 50 has convex surfaces allowing the protruding body of the barb 24 to be free prior to retraction of the plunger 13,

Region 51 is the smallest diameter of the bore 44 in the needle hub 15 and prevents the spring clip 22 from expanding and riding over the stem 23 at the end of the plunger 13 as shown in FIG. 12. The conical vertical wall 44 allows the retraction of the plunger 13 and the needle hub 15 to take place together without separation occurring.

With the specially designed shape of the bore 44, experience has shown that failures are reduced.

FIGS. 11 and 12 also exemplify the use of the clip of the invention with a needle which in this instance would be regarded as in non replaceable. However as discussed above the syringe 11 may also have an exchange needle. In either case an O-ring 52 is shown located about the hub 15 which provides sealing against leakage between the hub 15 and a gland or the needle barrel itself, if fitted directly therein.

Returning to FIG. 1, a pin 39 is also located in hole 40 in the upper region of plunger 13. Pin 39 acts as a depth stop to prevent accidental engagement of the plunger 13 with the needle hub 15 prior to use. When the syringe 11 is about to be used, the pin 39 can be easily depressed so as to allow full travel of the plunger 13 in the barrel 12 for mating engagement with the needle hub 15 as described above.

Thus in use, after an injection has been completed, the plunger 13 is pushed home to engage with the needle hub 15 by means of the action of the barbed spring clip 22 mating with the needle hub 15.

When the plunger 13 is pulled back, after the needle 14 is removed from the patient, the stainless steel barb 34 on the plunger 13 locks into the needle hub 15 and retracts the needle hub 15 and thus the needle 14 into the body 12 of the syringe 11 rendering the needle 14 safely stored from human harm. The separation of needle 14 from the plunger 13 cannot be achieved and when the plunger 13 is retracted and locked into the threaded split bush 28 (as in the earlier FIGS. 1 and 2) and the plunger 13 is snapped off, the syringe 11 is then locked and is not able to be used again.

The plunger 13 is then snapped off at break point 27. With the plunger 13 disabled and the needle 14 retracted inside the body 12 of the syringe 11, it is rendered totally useless and may then be disposed of safely.

It will be appreciated by those skilled in the art that many modifications and variations may be made to the embodiments described herein without departing from the spirit or scope of the invention.

Throughout the specification the word “comprise” and its derivatives are intended to have an inclusive rather than exclusive meaning unless the context requires otherwise.

1. A safety syringe incorporating a retractable needle wherein the syringe has a syringe barrel which has a plunger slideably located therein, with piston means located towards the end of the plunger remote from the end of the barrel from which the plunger extends, a needle having a needle hub integral with the needle, wherein the plunger has engagement means in the form of a cylindrical clip fitted about a shank or a stem on the end of the plunger having barbed means associated therewith and located on its end to engage with the needle hub in order to retract the needle from an in use position into the syringe barrel after the syringe has been used.

2. A safety syringe according to claim 1, in which the needle hub is held directly by the barrel of the syringe.

3. A safety syringe according to claim 1, in which the needle hub is held in a removable gland prior to be being withdrawn, to provide an exchange needle facility.

4. A safety syringe according to claim 1, in which the gland is fitted to the end of the barrel by threaded means.

5. A safety syringe according to claim 1, in which the clip is provided with one or more barbs.

6. A safety syringe according to claim 6, in which each one or more barbs is preferably formed from a portion of the wall of the clip which is cut so as to form a flap which when bent provides a barbed element.

7. A safety syringe according to claim 7, in which the barb is formed so as to have a sharp point.

8. A safety syringe according to claim 7, wherein a dimple is formed along a portion of the fold line by which the flap is bent to form the barb.

9. A safety syringe according to claim 8, wherein a corresponding groove is provided in the inner region of the needle hub to accommodate the dimple.

10. A safety syringe according to claim 1, in which the barrel is provided with a locking bush, which when the plunger is fully retracted with the needle attached, the plunger is prevented from being removed from the barrel or depressed again, thereby rendering the syringe safe and unusable.

11. A safety syringe according to claim 1, in which a break point is provided on the plunger so that the plunger may be snapped off retaining the needle safely within the syringe barrel after use.

12. A safety syringe according to claim 1, in which the needle is protected by a needle cap prior to use.

13. A safety syringe according to claim 3, in which the needle is protected by a needle cap prior to use and the gland is held in the needle cap by interference fit.

14. A safety syringe according to claim 14, in which there are matching splines on the outside of the needle gland that match with splines on the inside of needle cap.

15. A safety syringe according to claim 14 in which the needle hub is pushed into the needle gland and is held in position by an interference fit.

16. A safety syringe according to claim 15, in which sealing is achieved by the use of an O-ring located between the needle hub and the gland.

17. A safety syringe having a barrel with a plunger located therein and a retractable needle which is located directly in the barrel of the syringe or in a gland for removable retention in body of the syringe, the plunger having built into its end a clip to engage with the hub of a needle after the plunger has expelled fluid from the syringe and thus after completing an injection, so that when the two mating parts, being the end of the hub with clip attached and the needle hub, are pushed together, the plunger is then pulled back causing the used needle hub inclusive of the needle with which it is integral, to come out of the needle gland or the body of the syringe as the case may be and is withdrawn into the body of the syringe.

18. A safety syringe according to claim 17, in which the plunger has a snap point created by way of reduced diameter near the piston, so that when the plunger is snapped off, the syringe is rendered inoperative.

19. A safety syringe according to claim 18, wherein a split threaded bush is located around the body of the plunger and pushed into the barrel end and is held in position by way of an interference fit in a matching groove, the split bush acting as a locking means to secure the plunger against further use.

20. A safety syringe according claim 1, in which the clip is a stainless steel clip pressed from sheet metal and pushes over a stem or shaft on the end of the plunger.

21. A safety syringe according to claim 20, wherein the stainless steel clip is made in the form of a “C” section that clamps around the stem or shaft approximately 5 mm long with a barb being formed on one side to interact with the inner wall of the needle hub at the completion of injection.

22. A safety syringe according to claim 21, wherein after the needle is removed from a patient following an injection, the plunger is pulled back, the stainless steel barb on the plunger having locked into the needle hub causing the needle hub and thus the needle to retract into the body of the barrel thereby rendering the needle safely stored from human harm.

23. A safety syringe according to claim 21, in which the stem or shaft on the plunger has an interference fit to the inside of the needle hub which makes it obvious to a user when filling the syringe prior to injection at which point the plunger initially “bottoms” in the syringe barrel without engaging the needle hub, but whereas after injection a more powerful stroke is utilised to overcome the initial resistance in order to actually lock the needle hub onto the plunger.

24. A safety syringe according to claim 1, in which a depth stop is provided on the plunger to prevent the plunger from coming into mating contact with the needle hub until it is required to do so, that is to say until the completion of the injection.

25. A safety syringe according to claim 1, in which the cavity in the needle hub which receives the stem of the plunger to which the barbed clip attached, is shaped so as to improve the efficiency of the barb in engaging with the hub.

26. A safety syringe according to claim 26 in which the needle hub is provided with a cavity or shaped bore which has a first region of sufficient diameter to provide clearance for the plunger and spring clip along with the protruding barb on the clip, a second region which is tapered in order to cause the barb on the spring clip to bite into the wall of the needle hub, a third region which allows the barb on the spring clip to settle in to the correct position prior to retraction of the plunger/needle hub and allows for any variation in length to be and accommodated, a fourth region which has convex surfaces allowing the protruding body of the barb to be free prior to retraction of the plunger and a region having is the smallest diameter of the cavity or bore in the needle hub which prevents the spring clip from expanding and riding over the stem at the end of the plunger.

27. (canceled)
